(19) United States
(12) Patent Application Publication
(10) Pub. No.: US 2025/0213825 A1
(43) Pub. Date: Jul. 3, 2025

(54) ANTEGRADE FEMORAL ARTERY ENTRY DEVICE AND SHEATH

(71) Applicant: THE COOPER HEALTH SYSTEM, Camden, NJ (US)

(72) Inventors: Jeffrey P. CARPENTER, Moorestown, NJ (US); Katherine K. MCMACKIN, Ardmore, PA (US)

(21) Appl. No.: 18/852,162

(22) PCT Filed: Apr. 5, 2023
(86) PCT No.: PCT/US2023/017519

§ 371 Date: Sep. 27, 2024

Related U.S. Application Data
(60) Provisional application No. 63/327,414, filed on Apr. 5, 2022.

(51) Int. Cl. A61M 25/09

(52) U.S. Cl. CPC A61M 25/09041; A61M 2025/09116; A61M 2025/09125

(57) ABSTRACT

A sheath delivery system comprises an access device for insertion of one or more wires to extend in first and second directions within a blood vessel. The access device can include an elongated member with a lumen between a distal tip and proximal end, the lumen oriented to exit a patient, a footplate having a bore extending therethrough the footplate and configured to translate within the lumen between an initial state along the lumen and a rotated state anchoring the access device within a blood vessel, a first wire port through which a first wire enters and advances past the footplate into the blood vessel in a first direction, and a second wire port configured to guide a second wire into the lumen so that the second wire passes through the lumen and through the hollow bore of the footplate such that the second wire advances in the second direction.

### CROSS REFERENCE TO RELATED APPLICATIONS

This application claims the benefit of U.S. Provisional Application No. 63/327,414, “Antegrade Femoral Artery Entry Device And Sheath” (filed Apr. 5, 2022), the entirety of which application is incorporated by reference herein for any and all purposes.

### TECHNICAL FIELD

The present disclosure relates generally to an artery entry device and sheath system, and more particularly to an artery entry device via an antegrade approach and curved sheath for use therein.

### BACKGROUND

The traditional method for doing an angiogram from an antegrade approach requires the operator to contend with the patient's body habitus when trying to access the femoral artery. The abdominal wall, which can include an overlying pannus, can make access very difficult. Access can be easily lost, which can result in the patient bleeding or inability to complete the procedure.

If antegrade access has been successfully achieved, the operator still has to contend with the patient's body habitus to complete the angiogram. Wires, catheters, balloons, and stents must all go up over the abdomen and avoid contamination by the patient's face. To maintain a sterile field, the devices are curved away from the patient which makes exchanges of devices over the wire difficult. During exchanges, wires can be accidently pulled back, losing ground which may not be able to be crossed again, leading to an inability to complete the procedure. Accordingly, there is a long-felt need in the field for devices and methods for improved devices and methods for achieving antegrade and retrograde direction of wires and other instruments.

### SUMMARY

In meeting these long-felt needs, the present disclosure provides, inter alia, a new device that (1) serves as a means of rotating the position and angle of a wire and sheath from a retrograde to antegrade approach during an angiogram, to facilitate easy access; and (2) after rotation to an antegrade orientation, has a sheath component that allows for more ergonomic completion of the angiogram. The access piece is a device with a footplate aligned along the antegrade/retrograde axis. The device has a hollow lumen exiting the antegrade direction and an entry port in the back end of the device for wire insertion. The second piece can be a sheath with a curvature outside the body to direct the wires/catheters/balloons/stents away from the patient and towards the scrub table. The sheath also has anchors to secure the sheath to the patient.

According to certain aspects of the present disclosure, systems and methods are disclosed for an artery entry device and sheath system.

A sheath delivery system comprises an access device for insertion of one or more wires to extend in generally opposed first and second directions within a blood vessel. The access device comprises an elongated member with a lumen extending between a distal tip and a proximal end, the lumen oriented to exit a patient in a direction, the direction optionally being a retrograde direction; a footplate having an axis point about which the footplate pivots, the footplate configured to pivot within the lumen between an initial state that generally lies along the lumen and a rotated state, the footplate anchoring the access device within a blood vessel when the footplate is in the rotated state; a first wire port through which a first wire enters the lumen and advances past the footplate into the blood vessel, so that the first wire advances through the blood vessel in a first direction; and a second wire port configured to guide a second wire into the lumen so that the second wire passes through the lumen such that the second wire advances through the blood vessel in a second direction. In some embodiments, the footplate comprises a bore extending therethrough such that the second wire passes through the bore of the footplate when the footplate is in the rotated state and the second wire advances in the second direction. In some embodiments, the sheath delivery system further comprises a pivoting conduit in communication with the second wire port, the pivoting conduit configured to pivot within the lumen between an initial state that generally lies along the lumen and a rotated state, and the pivoting conduit configured to guide the second wire so that said wire advances in the second direction when the pivoting conduit is in the rotated state. In some embodiments, the sheath delivery system further comprises a conduit in communication with the second wire port, the conduit configured to receive and guide the second wire so that said wire advances in the second direction. In some embodiments, the first direction is a retrograde direction. In some embodiments, the second direction is an antegrade direction. In some embodiments, the sheath delivery system further comprises a footplate control lever coupled to the footplate and configured to effect translation of the body about the axis point. In some embodiments, the sheath delivery system further comprises a depth indicator. In some embodiments, the sheath is a curved sheath.

A sheath for use with a sheath delivery system can comprise an elongated lumen with a distal tip and an open proximal end; a curved section located proximal the distal tip such that the curved section does not advance past an entry point into a patient; and a plurality of suture wings located along the curved section, wherein a suture wing is configured to receive a suture that fastens the sheath to a skin surface adjacent to the entry point into the patient, such that the curved section of the sheath directs a wire exiting the sheath from the proximal end away from the patient.

A method of using a sheath delivery system, the method comprising inserting a first wire into a blood vessel; inserting an access device into the blood vessel over the first wire, the access device having a footplate and a depth indicator; receiving an indication from the depth indicator that the access device is sufficiently inserted in the blood vessel to engage the footplate; advancing the first wire through the blood vessel in a first direction; pivoting the footplate so that the footplate is aligned with an antegrade/retrograde axis within the blood vessel, thereby anchoring the access device in the blood vessel; and advancing a second wire through the access device and through a bore in the footplate into the blood vessel in a second direction.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with this description, serve to explain the principles of the disclosed embodiments.

FIG. 1 is an exemplary embodiment of an access device for insertion of one or more wires, according to the present disclosure.

FIG. 2 is an exemplary embodiment of insertion of an access device for one or more wires, according to the present disclosure.

FIG. 3 is an exemplary embodiment of deployment of an access device for one or more wires, according to the present disclosure.

FIG. 4 is an exemplary embodiment of removal of an access device for one or more wires, according to the present disclosure.

FIG. 5 is an exemplary embodiment of a sheath for use with an access device, according to the present disclosure.

FIG. 6 is an exemplary setting for use of a sheath for use with an access device, according to the present disclosure.

FIG. 7 is a flowchart illustrating a method of using an access device, according to the present disclosure.

### DETAILED DESCRIPTION

Reference will now be made in detail to the exemplary embodiments of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, system, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead can be performed in a different order or in parallel. As used herein, the term “exemplary” is used in the sense of “example,” rather than “ideal.” Moreover, the terms “a” and “an” herein do not denote a limitation of quantity, but rather denote the presence of one or more of the referenced items.

Using an antegrade approach for an angiogram typically requires the operator to contend with the patient's body habitus when trying to access the femoral artery, a common access point for insertion. The abdominal wall, which can include an overlying pannus, can make access very difficult. Antegrade access can be easily lost, which can result in the patient bleeding or inability to complete the procedure. If antegrade access has been successfully achieved, the operator still has to contend with the patient's body habitus to complete the angiogram. Wires, catheters, balloons, and stents must all go up over the abdomen and avoid contamination by the patient's face. To maintain a sterile field, the devices are curved away from the patient which makes exchanges of devices over the wire difficult. During exchanges, wires can be accidently pulled back, losing ground which can not be able to be crossed again, leading to an inability to complete the procedure. Additionally, multiple access points may be necessary for complex cases.

What is needed is a multi-part device allowing a user easier access to the femoral artery for insertion in the antegrade direction. The first part of the device, which facilitates access, allows the user to access the artery in the tradition manner, avoiding the issues with the pannus, and then rotate the sheath from a retrograde to an antegrade approach. This allows the user to access a vessel by the means with which he or she is most comfortable and that is used most often (a retrograde approach), and then switch to the antegrade approach. The second part of the device, the sheath used for the angiogram itself, allows the user a more ergonomic positioning of the operator in relation to the x-ray beam. The sheath also provides a pivot-point for the wire/catheter/balloon/stent exchanges, removing the need for the operator to risk contamination by the patient's face or for the operator to be directly under the x-ray beam.

The two separate pieces of the device 1) serve as a means of rotating the position and angle of a wire and sheath from a retrograde to an antegrade approach during an angiogram, to facilitate easy access; and 2) after rotation to an antegrade orientation, include a component that is a sheath that allows for more ergonomic completion of the angiogram. The access piece (first piece) is a device with a footplate aligned along the antegrade/retrograde axis. It has a hollow lumen exiting the antegrade direction and an entry port in the back end of the device for wire insertion. The second piece is a sheath with a curvature outside the body to direct the wires/catheters/balloons/stents away from the patient and towards the scrub table. It also has anchors to secure the sheath to the patient.

The device facilitates the performance of angiograms via an antegrade approach. The angiogram process is improved by simplifying the access of the blood vessel, allowing for more optimal wire placement, providing a pivot point to diminish wire loss during the angiogram itself. The device also allows the operator to be further from the source of radiation and keep his/her hands further from the radiation field.

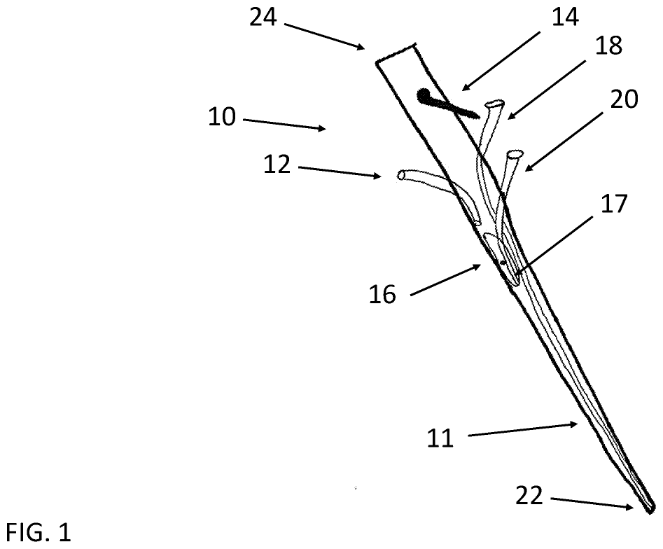

FIG. 1 is an exemplary embodiment of the access device for insertion of one or more wires. The access device 10 is an elongated lumen 11 extending between a proximal end 24 with an entry port and a distal end 22.

The access device 10 has a footplate control lever 14 near the proximal end 24, by which a user can control the footplate 16. The footplate 16 has a central point about which the footplate 16 can rotate or pivot. The footplate 16 has a body through which a central bore 17 extends. The footplate 16 is configured to pivot within the lumen 11 between an initial state where the footplate 16 is generally parallel to the lumen 11, and a rotated state, where the footplate 16 is generally perpendicular to the lumen 11.

The access device 10 can include a number of ports extending from the lumen 11. The ports include a depth indicator 12, a retrograde wire port 18, and an antegrade wire port 20. The depth indicator 12 indicates when the distal end 22 of access device 10 is inserted into the artery, and allows for arterial blood to exit the access device 10 through the depth indicator 12. Once blood has exited through the depth indicator 12, a user can continue the process of inserting the footplate 16 into the artery. Retrograde wire port 18 allows for a retrograde wire to advance through the access device 18 and be inserted into the artery. Access device 10 is placed over the retrograde wire in a standard fashion. Antegrade wire port 20 allows for an antegrade wire to be inserted into the access device 10 and through the central bore 17 of the footplate 16 into the artery.

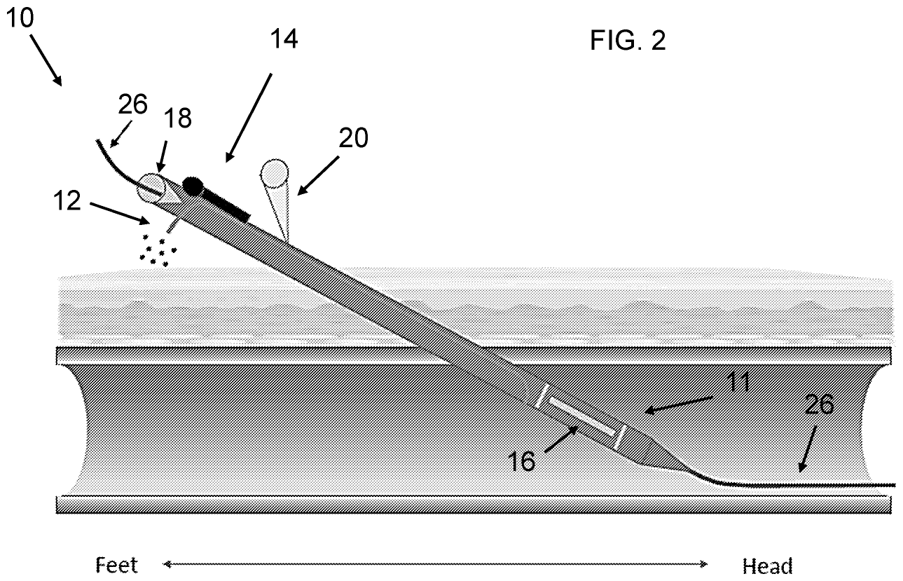

FIG. 2 is an exemplary embodiment of insertion of the access device for one or more wires. A retrograde wire 26 is advanced through the artery, exiting the access device 10 through the retrograde wire port 18. The access device 10 is inserted into the artery to a depth where bleeding occurs, such that blood exits the depth indicator 12. This indicates that there is adequate depth to deploy the footplate 16. The footplate 16 can begin in a position wherein the footplate 16 is flush with or enclosed within lumen 11 of the access device 10.

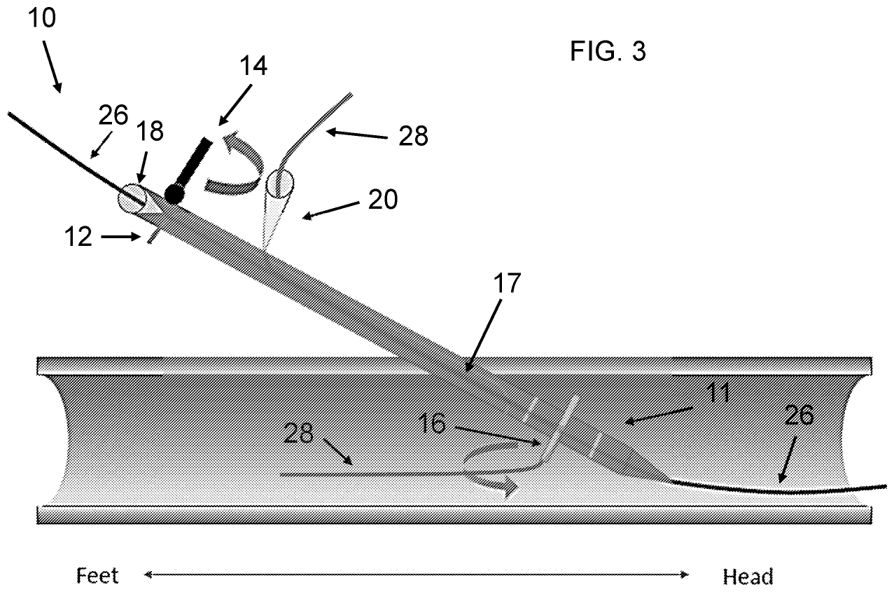

FIG. 3 is an exemplary embodiment of deployment of the access device for one or more wires. The user actuates the footplate control lever 14 to deploy the footplate 16. The footplate 16 is rotated around a central axis to be generally parallel to the artery and the access device 10 is pulled towards the user, such that the footplate 16 acts as an anchor for the access device 10 in the artery. A second wire (antegrade wire 28) is inserted through the antegrade wire port 20 to move through a conduit of the access device 10 and through the center bore 17 of the footplate 16 to enter the artery. The conduit can include the lumen 11 with an access point in communication with the antegrade wire port 20. The conduit can include a separate conduit distinct from lumen 11 that passes through the core of access device 10. The retrograde wire 26 is still inserted into the artery, but does not necessarily impact or contact the footplate 16 and/or the antegrade wire 28.

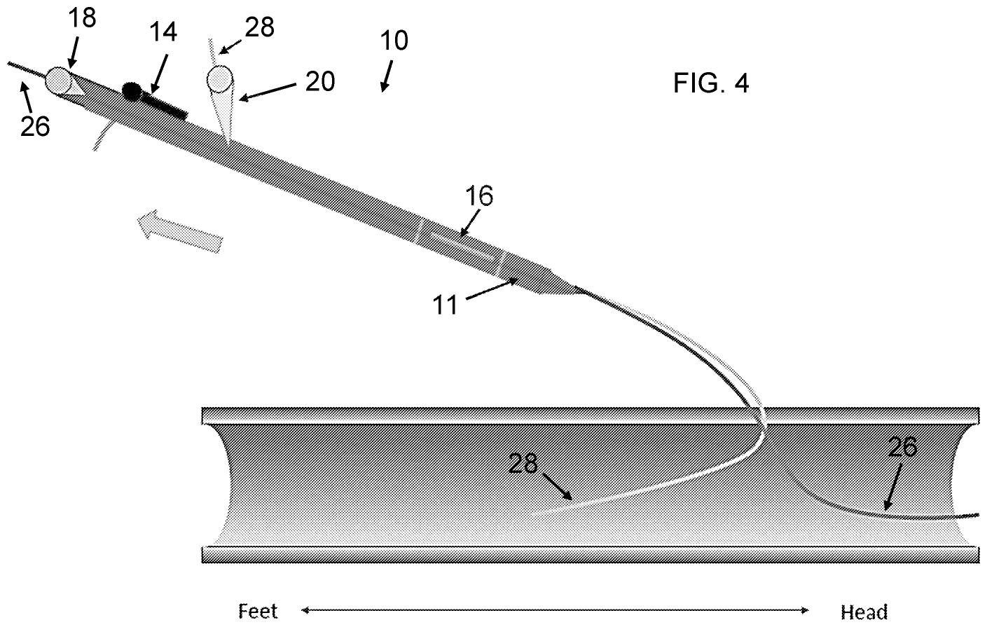

FIG. 4 is an exemplary embodiment of removal of the access device for one or more wires. The user once again moves the footplate control lever 14, this time to rotate the footplate lever back to a position where the body of the footplate lever is parallel to the lumen 11 and can, in some embodiments, be retained within the lumen 11. The antegrade wire 28 remains running through the bore 17 (shown in FIG. 4) of the footplate 16 and in the artery, where it can be advanced through the artery in the antegrade direction. Likewise, the retrograde wire 26 is still in the artery, advanced in the retrograde direction. The user can pull the access device 10 away from the artery at this point, such that distal end 22 is outside of the arterial access point.

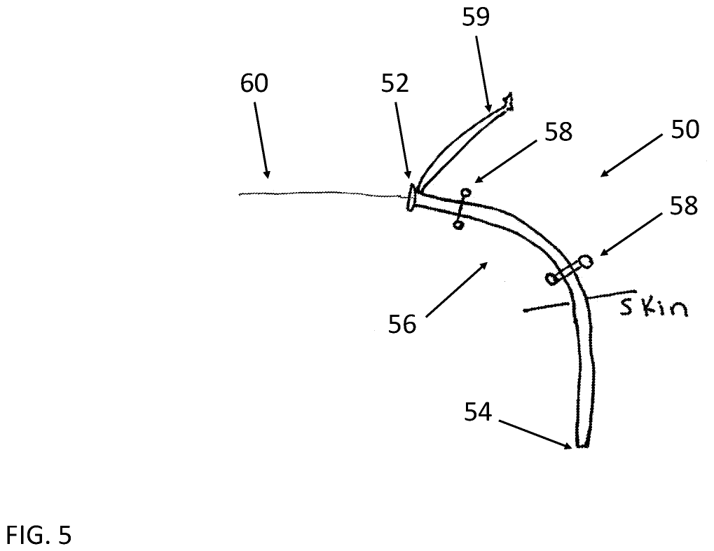

FIG. 5 is an exemplary embodiment of a sheath for use with an access device such as access device 10. The sheath 50 can have a hollow body with a curved portion 56, a distal end 54 and a proximal end 52. Side port 59 can be used to inject contrast dye for performing an angiogram, heparinized saline to ensure there is not thrombus formation, or to draw blood. If a fluid is later infused through the sheath, the fluid is hooked to side port 59. The curved portion 56 of the body is proximal of the portion of the sheath 50 inserted into the patient. The curved portion 56 of the body allows for the user to insert a wire, such as antegrade wire 60, without having to accommodate or navigate a patient's abdomen. The curved portion 56 can also have a number of suture wings 58. The suture wings 58 are present along the body of the sheath and have a number of holes for suturing the sheath body to the patient. The curved portion 56 can have a curvature ranging from 90 to 180 degrees. The sheath itself can come in a variety of dimensions, e.g., between 4 Fr and 8 Fr.

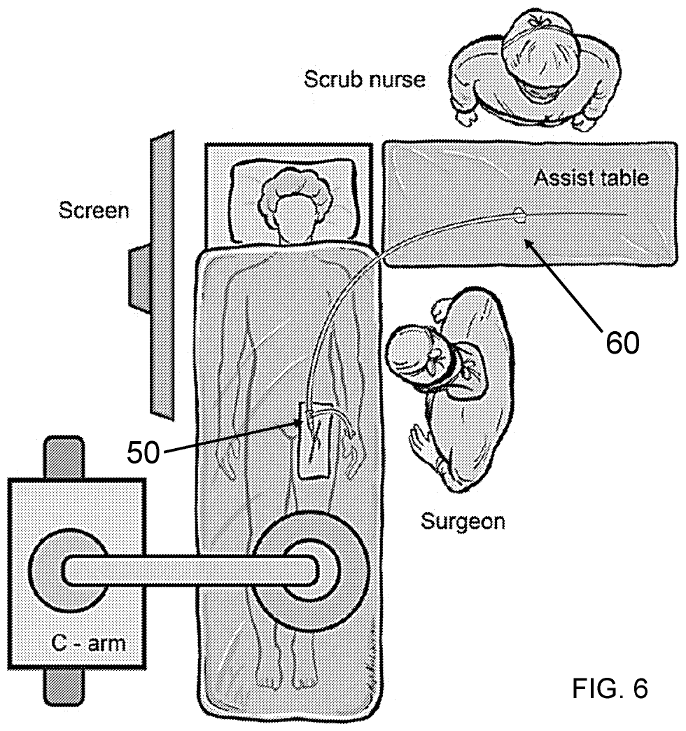

FIG. 6 is an exemplary embodiment of the environment in which an access device and sheath can be used. Within the operating area of a surgical room, a patient can be placed on the table for use with imaging equipment. The sheath 50 can be inserted into a patient's femoral artery, and the curved portion 56 can allow for the antegrade wire 60 to extend towards a table instead of over the patient's body. Therefore, the users are able to control the wire 60 much more effectively within this setting. This also allows for improved sterility, as the wire does not extend over the patient's face.

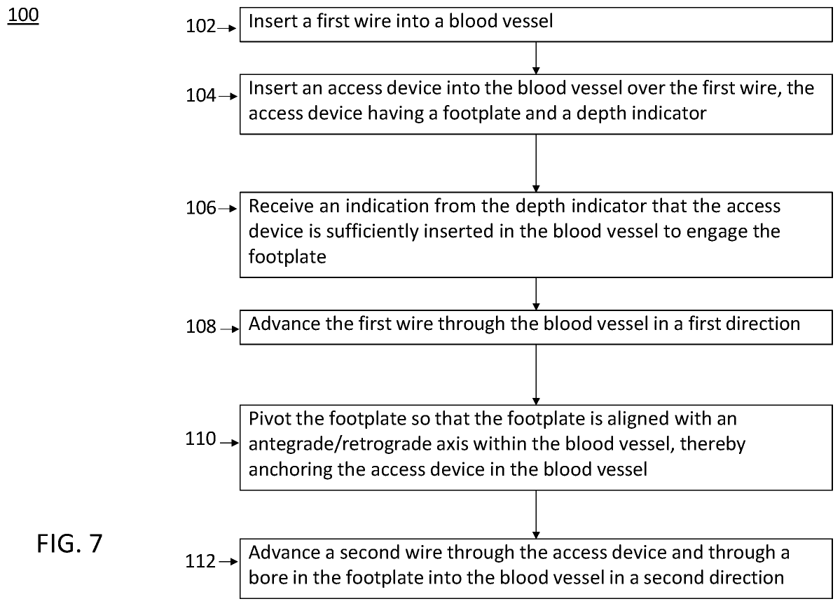

FIG. 7 is a flowchart illustrating an exemplary method of using an access device to advance one or more wires in a blood vessel. The exemplary method 100 of using the access device, such as access device 10, can include one or more of the following steps and can be performed by a user in preparation for an angiogram or other procedure.

In step 102, the method can include inserting a first wire into a blood vessel. The first wire can be inserted into the blood vessel in the retrograde direction. The blood vessel can be, for example, the femoral artery. Alternatively, in some embodiments, the first wire can be inserted into the blood vessel in the antegrade direction.

In step 104, the method can include inserting an access device into the blood vessel over the first wire, the access device having a footplate and a depth indicator. The footplate is controlled by a footplate lever on the body of the access device, by which the user can control the rotation of the footplate around a point on the footplate. The access device additionally has a number of ports between a proximal end and a distal tip, through which additional wires, stents, catheters, or other devices can be inserted into the blood vessel as needed.

In step 106, the method can include receiving an indication from the depth indicator that the access device is sufficiently inserted in the blood vessel deep enough to engage the footplate. The depth indicator comprises an exit port on the body of the access device, where blood exits the access device after traveling through the portion of the access device that is inserted into the blood vessel.

In step 108, the method can include advancing the first wire through the blood vessel in a first direction. The first wire is advanced in the standard fashion along an antegrade/retrograde axis within the blood vessel, in either the antegrade or retrograde direction as the user prefers.

In step 110, the method can include pivoting the footplate so that the footplate is aligned with the antegrade/retrograde axis within the footplate, thereby anchoring the access device in the blood vessel. To pivot the footplate, the user engages the footplate lever so the footplate rotates against (and can be flush with) the wall of the blood vessel. The access device is then anchored into the blood vessel such that the user can insert another wire into the blood vessel.

In step 112, the method can include advancing a second wire through the access device, through a bore in the body of the footplate, into the blood vessel in a second direction. The second wire is inserted into the access device through another port into the lumen of the access device. The second wire can be advanced in the antegrade direction through the blood vessel. Alternatively, in some embodiments, the second wire can be inserted into the blood vessel and advanced in the retrograde direction.

### Embodiments

The following Embodiments are illustrative only and do not limit the scope of the present disclosure or the appended claims. Any part or parts of any one or more Embodiments can be combined with any part or parts of any one or more other Embodiments.

Embodiment 1. A sheath delivery system, comprising an access device for insertion of one or more wires to extend in generally opposed first and second directions within a blood vessel. The access device can include an elongated member with a lumen extending between a distal tip and a proximal end, the lumen oriented to exit a patient in a direction, the direction optionally being a retrograde direction, a footplate having an axis point about which the footplate pivots, the footplate configured to pivot within the lumen between an initial state that generally lies along the lumen and a rotated state. The footplate can anchor the access device within a blood vessel when the footplate is in the rotated state, a first wire port through which a first wire enters the lumen and advances past the footplate into the blood vessel, so that the first wire advances through the blood vessel in a first direction, and a second wire port configured to guide a second wire into the lumen so that the second wire passes through the lumen such that the second wire advances through the blood vessel in a second direction.

Embodiment 2. The sheath delivery system of Embodiment 1, wherein the footplate comprises a bore extending therethrough such that the second wire passes through the bore of the footplate when the footplate is in the rotated state and the second wire advances in the second direction.

Embodiment 3. The sheath delivery system of Embodiment 1, further comprising a pivoting conduit in communication with the second wire port, the pivoting conduit configured to pivot within the lumen between (i) an initial state that generally lies along the lumen and (ii) a rotated state, and the pivoting conduit configured to guide the second wire so that said wire advances in the second direction when the pivoting conduit is in the rotated state. The pivoting conduit can pivot in concert with pivoting of the footplate; in some embodiments, a mechanism pivots the pivoting conduit and the footplate in concert. This is not a requirement, however, as the pivoting conduit can pivot independently of the footplate.

Embodiment 4. The sheath delivery system of Embodiment 1, further comprising a conduit in communication with the second wire port, the conduit configured to receive and guide the second wire so that said wire advances in the second direction. The conduit can be nonrotatable.

Embodiment 5. The sheath delivery system of any one of Embodiments 1-4, wherein the first direction is a retrograde direction. As described elsewhere herein, the first direction can be an antegrade direction.

Embodiment 6. The sheath delivery system of any one of Embodiments 1-5, wherein the second direction is an antegrade direction. As described elsewhere herein, the second direction can be a retrograde direction.

Embodiment 7. The sheath delivery system of any one of Embodiments 1-6, further comprising a footplate control lever coupled to the footplate and configured to effect translation of the footplate about the axis point.

Embodiment 8. The sheath delivery system of any one of Embodiments 1-7, further comprising a depth indicator.

Embodiment 9. The sheath delivery system of any one of Embodiments 1-8, further comprising a curved sheath.

Embodiment 10. A sheath for use with a sheath delivery system, comprising an elongated lumen with a distal tip and an open proximal end, a curved section located proximal the distal tip such that the curved section does not advance past an entry point into a patient, and a plurality of suture wings located along the curved section, wherein a suture wing is configured to receive a suture that fastens the sheath to a skin surface adjacent to the entry point into the patient, such that the curved section of the sheath directs a wire exiting the sheath from the proximal end away from the patient.

Embodiment 11. The sheath of Embodiment 10, wherein the curved section comprises a two-dimensional curve in an x-y plane.

Embodiment 12. The sheath of any one of Embodiments 10-11, wherein the curved section comprises a three-dimensional curve along a z-axis.

Embodiment 13. A method of using a sheath delivery system, comprising: inserting a first wire into a blood vessel, inserting an access device into the blood vessel over the first wire, the access device having a footplate and a depth indicator, receiving an indication from the depth indicator that the access device is sufficiently inserted in the blood vessel to engage the footplate, advancing the first wire through the blood vessel in a first direction, pivoting the footplate so that the footplate is aligned with an antegrade/retrograde axis within the blood vessel, thereby anchoring the access device in the blood vessel, and advancing a second wire through the access device and through a bore in the footplate into the blood vessel in a second direction. A sheath delivery system can be, e.g., a sheath delivery system according to any one of Embodiments 1-9, and/or a sheath according to any one of Embodiments 10-12.

Embodiment 14. The method of Embodiment 13, wherein the first direction is a retrograde direction. As described elsewhere herein, the first direction can be an antegrade direction.

Embodiment 15. The method of any one of Embodiments 13-14, wherein the second direction is an antegrade direction. As described elsewhere herein, the second direction can be a retrograde direction.

1. A sheath delivery system, comprising:
an access device for insertion of one or more wires to extend in generally opposed first and second directions within a blood vessel, the access device comprising:
an elongated member with a lumen extending between a distal tip and a proximal end, the lumen oriented to exit a patient in a direction, the direction optionally being a retrograde direction;
a footplate having an axis point about which the footplate pivots, the footplate configured to pivot within the lumen between an initial state that generally lies along the lumen and a rotated state, the footplate anchoring the access device within a blood vessel when the footplate is in the rotated state;
a first wire port through which a first wire enters the lumen and advances past the footplate into the blood vessel, so that the first wire advances through the blood vessel in a first direction; and
a second wire port configured to guide a second wire into the lumen so that the second wire passes through the lumen such that the second wire advances through the blood vessel in a second direction.

2. The sheath delivery system of claim 1, wherein the footplate comprises a bore extending therethrough such that the second wire passes through the bore of the footplate when the footplate is in the rotated state and the second wire advances in the second direction.

3. The sheath delivery system of claim 1, further comprising a pivoting conduit in communication with the second wire port, the pivoting conduit configured to pivot within the lumen between (i) an initial state that generally lies along the lumen and (ii) a rotated state, and the pivoting conduit configured to guide the second wire so that said second wire advances in the second direction when the pivoting conduit is in the rotated state.

4. The sheath delivery system of claim 1, further comprising a conduit in communication with the second wire port, the conduit configured to receive and guide the second wire so that said second wire advances in the second direction.

5. The sheath delivery system of claim 1, wherein the first direction is a retrograde direction.

6. The sheath delivery system of claim 1, wherein the second direction is an antegrade direction.

7. The sheath delivery system of claim 1, further comprising a footplate control lever coupled to the footplate and configured to effect translation of the footplate about the axis point.

8. The sheath delivery system of claim 1, further comprising a depth indicator.

9. The sheath delivery system of claim 1, further comprising a curved sheath.

10. A sheath for use with a sheath delivery system, the sheath comprising:
an elongated lumen with a distal tip and an open proximal end;
a curved section located proximal the distal tip such that the curved section does not advance past an entry point into a patient; and
a plurality of suture wings located along the curved section, wherein a suture wing is configured to receive a suture that fastens the sheath to a skin surface adjacent to the entry point into the patient, such that the curved section of the sheath directs a wire exiting the sheath from the open proximal end away from the patient.

11. The sheath of claim 10, wherein the curved section comprises a two-dimensional curve in an x-y plane.

12. The sheath of claim 10, wherein the curved section comprises a three-dimensional curve along a z-axis.

13. A method of using a sheath delivery system, the method comprising:
inserting a first wire into a blood vessel;
inserting an access device into the blood vessel over the first wire, the access device having a footplate and a depth indicator;
receiving an indication from the depth indicator that the access device is sufficiently inserted in the blood vessel to engage the footplate;
advancing the first wire through the blood vessel in a first direction;
pivoting the footplate so that the footplate is aligned with an antegrade/retrograde axis within the blood vessel, thereby anchoring the access device in the blood vessel; and
advancing a second wire through the access device and through a bore in the footplate into the blood vessel in a second direction.

14. The method of claim 13, wherein the first direction is a retrograde direction.

15. The method of claim 13, wherein the second direction is an antegrade direction.
